(19) United States
(12) Patent Application Publication
(10) Pub. No.: US 2025/0101359 A1
(43) Pub. Date: Mar. 27, 2025

(54) 3D HYDROGEL SYSTEM FOR CELL SCALE-UP AND BIOPRODUCTION

(71) Applicant: TheWell Bioscience Inc., Monmouth Junction, NJ (US)

(72) Inventor: Hongzhou HUANG, Monmouth Junction, NJ (US)

(73) Assignee: TheWell Bioscience Inc., Monmouth Junction, NJ (US)

(21) Appl. No.: 18/895,679

(22) Filed: Sep. 25, 2024

Related U.S. Application Data
(60) Provisional application No. 63/540,120, filed on Sep. 25, 2023.

(51) Int. Cl. C12M 1/12

(52) U.S. Cl. CPC C12M 25/14

(57) ABSTRACT

New and improved hydrogels and methods associated therewith including their use for cell cultures and various other biomedical applications are disclosed. Because the instant invention does not rely on two dimensional surface area for cell growth but uses a three dimensional model, the hydrogels as disclosed herein have improved properties that allow for improved cell growth, easier and better harvesting and isolation of cells and small molecule products such as exosomes, as well as higher yields that can be achieved less expensively.

### CROSS-REFERENCE TO RELATED APPLICATIONS

The present application claims priority under 35 USC 119(e) to U.S. Provisional Application No. 63/540,120 filed Sep. 25, 2023, the entire contents of which is incorporated by reference.

### FIELD OF THE INVENTION

The present invention relates to new and improved hydrogels and methods associated therewith including their use for cell cultures and various other biomedical applications. Because the instant invention does not rely on two-dimensional surface area for cell growth but rather uses a three-dimensional model, the hydrogels as disclosed herein have improved properties that allow for improved cell growth, easier harvesting, and higher yields of cell expansion and protein/exosome production.

### BACKGROUND OF THE INVENTION

The examination of biological phenomenon has been routinely explored in two-dimensional (2D) environments on unphysiologically rigid materials, such as glass coverslips.

Two-dimensional study resulted in a misunderstanding of many cellular behaviors, such as cell shape, proliferation, and differentiation. These 2D systems also prevented the proper examination of cellular responses to extracellular cues. Two-dimensional cell cultures rely on the ability of cells to sense their surroundings via dynamic leading-edge filopodia (A Arjonen, R. Kaukonen, and J. Ivaska, “Filopodia and adhesion in cancer cell motility,” Cell Adhesion and Migration, 2011), whereas three-dimensional cell cultures rely on other mechanisms.

Cells possess actin-rich apical and basal protrusions, such as invadopodia and podosomes, that allows cells to sense and respond to their environment in 3D. See M. Gimona, R. Buccione, S. A Courtneidge, and S. Linder, “Assembly and biological role of podosomes and invadopodia,” Current Opinion in Cell Biology, 2008; D. A Murphy and S. A Courtneidge, “The ‘ins’ and ‘outs’ of podosomes and invadopodia: Characteristics, formation and function,” Nature Reviews Molecular Cell Biology, 2011; and C. M. Gould and S. A. Courtneidge, “Regulation of invadopodia by the tumor microenvironment,” Cell Adhesion and Migration, 2014). Invadopodia are important regulators of extracellular matrix (ECM) remodeling during cancer metastasis, illustrating the advantage of studying cellular behaviors in 3D.

Three-dimensional (3D) cell culture models present a more accurate representation of the natural environment experienced by the cells in the living organism, which allows for intercellular interactions with more realistic biochemical and physiological responses. In 3D cell cultures, cells behave and respond more like they would in vivo to internal and external stimuli, such as changes in temperature, pH, nutrient absorption, transport, and differentiation. They also present different cellular signaling profiles. Therefore, scientists are shifting their focus from 2D to 3D cell cultures in the fields of drug screening, tissue engineering, preclinical study, cell therapy, and basic cell biological study. To mimic in vivo cell growing conditions, the reticulated structure of 3D scaffolds should be serialized, having a high water content, and having a number of other desirable characteristics.

Such other desirable characteristics include accurate 3D spatial support, suitable mechanical strengths, and facile transportation of oxygen, nutrients, waste, and soluble factors. Mild and cytocompatible conditions for sol-gel transformation are preferred, to ensure that cells survive comfortably during both cell encapsulation and isolation. Moreover, the injectable property of biomaterials used for 3D cell cultures is important for downstream applications, which include cancer therapy (xerography study for drug discovery), tissue regeneration, and 3D bio-printing.

The current materials for 3D cell cultures on the market can be classified as hydrogels, polymer matrices, hanging drop plates, low adhesion plates, micro-patterned surfaces, and magnetic levitations. With the development of 3D cell culture technology, hydrogels (or self-organized three-dimensional tissue cultures) have opened a new way for diseases to be studied and treated. The 3D assembling organoids contain multiple cell types that can be arranged similarly to the cells in a specific tissue. Organoids made from human cells or patient tissue become a valuable tool for medical research, and specifically for preclinical studies. To culture organoids, the hydrogel biomaterial plays an important role.

Hydrogel scaffolds have been demonstrated to be one of the most promising approaches to date in mimicking 3D cell culture. Hydrogels allow experimenters to encapsulate cells within the hydrogels or seed cells post-formation (see S. R. Caliari and J. A. Burdick, “A practical guide to hydrogels for cell culture,” Nature Methods, 2016). Organic microenvironments, e.g. collagen, expose cells to native culture systems possessing a variety of functional ligands (K. Wolf et al., “Collagen-based cell migration models in vitro and in vivo,” Seminars in Cell and Developmental Biology, 2009). Providing cells with a tissue-like environment permits the examination of unique cellular characteristics dependent upon the expression of specific cell surface receptors. However, while organic hydrogels produce a cell culture system that in many ways is similar to in vivo conditions, there are drawbacks (see S. R. Caliari and J. A. Burdick, “A practical guide to hydrogels for cell culture,” Nature Methods, 2016).

Most existing biomaterials (including hydrogel scaffolds) for 3D cell cultures are limited to unrealistic physiological conditions (e.g., poor scaffold structure, unwanted growth factors, and undesirable pH or temperature of pre-gel solution), complex operating steps for cell encapsulation, difficulties for cell isolation from the culture scaffold, and limited product reproducibility. In addition, injectable properties, such as shear-thinning and rapid recovery of physical strength, in currently marketed hydrogel materials is very rare. These drawbacks not only affect the data generated from these 3D cell culture technologies, but they also limit applications of this technology on downstream analysis and clinical applications.

Further, using native ECM in a cell culture is restrictive due to inconsistencies during production, the presence of growth factors, and inconsistencies of ligand bioactivity. To address these issues, more robust synthetic hydrogel systems are needed (see S. L. Bellis, “Advantages of RGD peptides for directing cell association with biomaterials,” Biomaterials, 2011; T. Maeda, K. Titani, and K. Sekiguchi, “Cell-adhesive activity and receptor-binding specificity of the laminin-derived YIGSR sequence grafted onto Staphylococcal protein a,” J. Biochem., 1994; and T. Y. Cheng, M. H. Chen, W. H. Chang, M. Y. Huang, and T. W. Wang, “Neural stem cells encapsulated in a functionalized self-assembling peptide hydrogel for brain tissue engineering,” Biomaterials, 2013).

The use of human pluripotent stem cells (hPSCs) in biomedical research is expanding exponentially. hPSCs provide an excellent system for studying basic human development and function, as well as providing a robust platform for testing stem cell-based therapies in laboratory and clinical settings. Recent advances in hPSCs procedures have opened the door for use in clinical trials. hPSCs have immense potential for use in regenerative medicine, as they may hold the key to reversing tissue damage caused by diseases and injuries. However, with this great potential comes the need for scaling up, as high throughput experiments are becoming commonplace due to the ever-increasing demand for more and better data.

The number of adults that are 60 and above is estimated to reach 1.4 billion by 2030. 85% of this population is expected to have at least one chronic illness, meaning that there is and there will continue to be a constant need to tap into different therapeutic methodologies. People have postulated that stem cell usage will at least to some extent serve as a partial substitute for medical and surgical procedures. MSCs hold an edge over other stem cells as they are immune privileged, enabling them to be used for transplantation. These cells secrete factors that can enhance and help repair tissue, and also possess anti-inflammatory effects. Diseases that are expected to require large quantities of MSCs include autoimmune, inflammatory, and metabolic diseases. The current global market size of MSCs was USD 2.7 billion in 2021, but is expected to achieve a level USD 6.1 billion by 2028, (i.e., a 12.6% growth rate).

To unlock the full potential of MSCs, large-scale manufacturing is the key. An average dose size needed for clinical therapy can range from 0.5 to 5×10⁶ cells/kg of body weight (the minimally effective dose (MEDs) range using MSC therapy is about 100 to 150 million MSCs/patient). The average dosing of MSC's needed per patient group is from 10¹² to 10¹³ cells. In a successful expansion of MSCs, cell quality needs to be maintained throughout the entire expansion process while obtaining large quantities of these cells. 2D planar substrates are somewhat conducive to expanding MSCs in lab settings, but these substrates are unable to easily achieve commercially relevant batch sizes due to the necessity of large space requirements, as well as the impracticality of handling vast volumes of T-flasks. Bioreactors are the preferred technology used to address the limitations of 2D cell culture, and microcarrier use is the currently preferred option to scale up adherent cells in a suspension bioreactor. However, microcarrier systems still rely on the surface area of the polymer matrix. They also require intense agitation rates to maintain cell suspensions, which reduces the functionality and viability of the cells while also causing significant challenges for cell harvesting and downstream processing.

Current stem cells and most adherent cell maintenance and expansion methods require plating cells on 2D matrix coated culture vessels with large surface areas, which can be time-consuming and expensive. Moreover, the matrix can be temperature sensitive. This can lead to an uneven coating of the culture vessel, thereby producing inconsistent stem cell colony adhesiveness and size. Therefore, 3D expansion systems offer a more consistent means of stem cell expansion. Besides expansion, stem cell researchers are discovering the many applications of 3D hPSC cultures. Perhaps, the most exciting of these is the development of sophisticated tissue organoids with laminar organization. In contrast, current 2D culture methods present some challenges to producing consistent, high-quality organoids.

Traditional methods have included “lifting” of stem cell colonies with harsh enzymes, such as trypsin, which can have deleterious effects on cell viability and produce stem cell spheroids with inconsistent sizes. Thus, current 2D stem cell methods often require long periods of troubleshooting to overcome these inconsistencies. Many established organoid protocols also require tissue-like precursors that need to be developed in a 2D system and after germ layer differentiation, the precursors must be lifted and embedded into a 3D matrix hydrogel. These multiple, complicated steps can ultimately result in inconsistent and malformed organoids.

For both small laboratories and large biotechnology companies, fast and dependable expansion of stem cell lines is becoming a critical need. However, current methods for scaling up stem cell populations require expensive and cumbersome equipment, such as shakers, spinning flasks, or bioreactors. These protocols also typically call for the use of microcarriers to aid in stem cell expansion. In many protocols, to ensure the formation of spheroidal stem cell aggregates, shakers or bioreactors are used at high speeds, which can elicit spheroidal shearing, resulting in impaired growth and cell viability.

Synthetic hydrogels permit greater reliability and control of experimental conditions. With greater hydrogel reproducibility, scientists can be assured that observed cellular behaviors and characteristics are consistent and accurate. Thus, a need exists for methods that relate to the at least partial purification of exosomes and/or cytokines and growth and at least certain types of cells using the hydrogels of the present invention.

### BRIEF SUMMARY OF THE INVENTION

The present invention relates to new and improved hydrogels and methods associated therewith including their use for cell cultures and various other biomedical applications. Because the instant invention does not rely on two-dimensional surface area for cell growth but uses a three-dimensional model, the hydrogels as disclosed herein have improved properties that allow for improved cell growth, easier harvesting, and higher yields.

In an embodiment, the present invention relates to using the 3D hydrogel system for biomanufacturing, including at least the following two processes: 1) cell scale-up, using the 3D hydrogel method for large-scale cell expansion and easy harvesting; and 2) applying the same culture method for bioproduction (i.e., not just expanding cells, but also using the scale-up method for protein and/or exosome production).

In an embodiment, any of a plurality of cell types can be used with the processes of the instant invention including but not limited to: MSC, IPSCs, ESCs, CSCs, HEK293, Osteoblasts, Hematopoietic stem cells, multipotency stem cells, neural stem cells, spermatogonial stem cells, adult stem cells, epithelial stem cells, chondrocytes, and/or unipotent stem cells.

### BRIEF DESCRIPTION OF THE SEVERAL VIEWS OF THE DRAWING

FIG. 1 shows an example of the rapid expansion rate and the health of cultured MSCs using the systems of the present invention over a period of 11 days.

FIG. 2 shows a bar graph of the hydrogels of the present invention (blue bars) vs. the microcarriers of the prior art (orange bars) over a period of 11 days.

FIG. 3 shows a bar graph showing the relative number of cells in a cell expansion of the present invention versus a 2D culture.

### DETAILED DESCRIPTION OF THE INVENTION

The present invention relates to new and improved hydrogels and methods associated therewith including their use for cell cultures and various other biomedical applications. Because the instant invention does not rely on two-dimensional surface area for cell growth but uses a three-dimensional model, the hydrogels as disclosed herein have improved properties that allow for improved cell growth, easier harvesting, and higher yields.

In an embodiment, the present invention relates to at least five different ways of using the hydrogels of the present invention. This includes a 3D culture, employing a 2D hydrogel coating, performing a static suspension culture, employing a hydrogel-cell bead, and using the hydrogels for animal injection.

In the 3D culture, the present invention encapsulates cells in the hydrogel matrix to promote cell-matrix and cell-cell interactions. In the 2D hydrogel coating methodology, one can control the substance properties on cell cultures by coating the hydrogel of the present invention with a 2D hydrogel coating. This methodology is ideal for cell submergence and for cell invasive studies. The static suspension culture methodology changes the viscosity of a cell medium to create a cell suspension environment for large-scale cell expansion. The hydrogel-cell bead methodology involves encapsulating cells in a hydrogel matrix by creating hydrogel-cell droplets. Finally, the animal injection methodology allows one to mix compounds or cells with injectable hydrogels to increase cell retention and viability of the cells for in vivo studies in the animals.

In an embodiment, the present invention relates to a hydrogel matrix that allows for reproducible scale up and systems that can be used for a variety of purposes. The synthetic hydrogels of the present invention permit greater reliability and control of experimental conditions. Because the hydrogel systems of the present invention can be reliably reproduced, the present invention should assure that a user of the systems of the present invention are observing cellular behaviors and characteristics that are not just consistent, but are also more precise and accurate.

In an embodiment, the present invention uses a state of the art biomimicking hydrogel system, which gives outstanding cell expansion rates and superior cell viability for 3D biomanufacturing processes. Compared to traditional 2D cultures or microcarrier systems, the hydrogel systems in use here possess spatial advantages that give the 3D matrix structures of the present invention advantageous cell-cell communication and cell-matrix interactions, while also attaining long-term cell culture capability.

In an embodiment, the present invention encapsulates cells in a hydrogel matrix as hydrogel-cell beads that are adaptable to 3D cell culture. Accordingly, the cells can be effectively manipulated with different matrix densities, degradabilities, and modified with active functional ligands to enhance and tailor the cells for specific cell growth. In an embodiment, to support cell growth in a hydrogel matrix, the hydrogel can be modified with different biological ligands such as peptides to increase the cell-matrix interactions to support cell growth. Moreover, the cells can also be quickly adapted for lab automation for large-scale 3D cell expansion. Another benefit is that the expanded cells can be easily harvested by quickly digesting the hydrogel matrix with a cell recovery solution for downstream processing. This technology is not only advantageous for cell scale-up, but should also prove to be superior for exosome production and other bioprocessing applications.

In an embodiment, the present invention relates to the field of 3D hydrogel cell cultures. In an embodiment, the present invention relates to xeno-free biomaterials solutions tailored at targeted 3D culture expansions in multiple cell lines. In a variation, the methods of the present invention lead to improved cell expansion. In an embodiment, Mesenchymal stem cells (MSCs) that are cultured in the hydrogel systems of the present invention have been shown to have significantly higher levels of cell viability, proliferation, and exosome production. In an embodiment, the hydrogel of the present invention provides a 3D spatial matrix for cell attachment and cell colony formation, which solves the surface area limitation issue of traditional 2D culture systems or microcarriers. The hydrogel matrix supports excellent nutrition and oxygen penetration for long-term cell culture with high cell viability, which is ideal for cell-based biomanufacturing without repetitive harvesting and passaging during cell expansion. By leveraging the tunability of the innovative hydrogels of the present invention, MSC scale-up can be accelerated with optimal hydrogel conditions and the hydrogel-based cell expansion can be automated. In an embodiment, the 3D hydrogel-based system for high-quality MSCs expansion can be attained on an industrial scale. MSCs cultured in the 3D systems of the present invention can expand to large numbers while maintaining phenotypical and functional properties, offering an advantage over the 2D culture or microcarrier systems of the prior art.

In an embodiment, the hydrogel systems of the present invention can be made from any of a plurality of materials. In an embodiment, the present invention relates to methods of MSC (mesenchymal stem cells) expansion and exosome production using the hydrogel systems of the invention. In an embodiment, the hydrogel beads (i.e., a layer of hydrogel) of the present invention can not only be used for MSC expansion and exosome production, but can also be used as a filter system for bioproduction, such as for the isolation, release and/or purification of the cells, and/or exosomes. In a general sense, hydrogel beads are a polysaccharide-containing hydrogel aggregation, which is capable of encapsulating cells inside them. In an embodiment, the present invention contemplates using the hydrogel as a filter system to support protein isolation and purification of proteins/exosomes, which is a benefit of the system beyond merely being used for cell expansion and cell production. In an embodiment, the cells that can be used for protein and/or exosome production and isolation including but not limited to MSC and HEK293 cells.

Exosomes are extracellular vesicles that are released from the cells upon fusion of an intermediate endocytic compartment. Exosomes deliver lipids, proteins, and nucleic acids to recipient cells when circulating in the extracellular space. Exosomes play a significant role in an immune response, in tumor progression, and in neurogenerative diseases. The present system can be used to manufacture exosomes, but the desired exosome can be more accurately made and selected because one has more accurate control over when the cell growth portion ends and the cell culture portion starts.

Thus, in an embodiment, the present invention relates to using the hydrogel beads of the present invention to not only at least partially purify the exosomes that are produced by the cells but to potentially perform experiments using the exosomes that are produced either with the hydrogel bead or potentially in downstream experiments after the exosomes have at least been partially purified.

In an embodiment, the hydrogel beads of the present invention can be used to produce and/or at least partially purifying cytokines. Cytokines are any of a number of substances, such as interferon, interleukin, and growth factors, which are secreted by certain cells of the immune system that will have an effect on other cells. Thus, in an embodiment, the present invention relates to using the hydrogel beads of the present invention to not only at least partially purify the cytokines that are produced by the cells but to potentially perform experiments using the cytokines that are produced either with the hydrogel bead or potentially in downstream experiments after the cytokines have at least been partially purified. Hydrogel beads may be formed by procuring a hydrogel or a hydrogel cell mixture that is added to a cell culture medium or to any crosslinking solution as droplets and form a bead structure.

In an embodiment, the hydrogels of the present invention can be modified with different biophysical and biological properties, including mechanical strength, degradability, and with functional ligands for cell-matrix interactions. The systems can also be supplemented with molecules such as growth factors, and other small molecules in the cell suspension before mixing with hydrogel for gel-cell mixture formation. In an embodiment, the biophysical and biological properties of the hydrogels of the present invention can be modified in a way that affects cell growth and allows one to manipulate hydrogel formation. In an embodiment, the hydrogel systems can be modified to more closely mimic in vivo behaviors. For example, the hydrogels can be modified with the RGD integrin-binding ligand, with collagen, laminin, and/or with MMPs (matrix metalloproteins).

In an embodiment, the present invention relates to modifying the dynamic mechanical properties of a xeno-free, tunable hydrogel system. In an embodiment, the diffusion properties are modified. In a variation, the properties of the hydrogel systems can be modified to support drug screening, toxicology assays, tissue engineering, regenerative medicine or other biomedical applications. The properties that can be adjusted include stiffness, density, porosity, molecular diffusion, ligand binding, as well as the composition. In an embodiment, the hydrogels of the present invention are tunable. In an embodiment, the rheological properties can be adjusted at different stages of the hydrogel formation including the dynamic shear thinning and/or recovery processes. The rheological properties can be examined for both their hydrogel formation and/or hydrogel stabilization properties. Furthermore, by varying concentrations and/or adjusting the ionic solution ratios, one can adjust the elastic modulus. Different salts can be added to test the rheological properties including using chloride salts such as calcium chloride and sodium chloride. In an embodiment, the rheological properties can be adjusted by adding different cell culture media, such as adding DMEM (Dulbecco's Modified Eagle Medium), DMEM/F12 (DMEM with supplements), RPMI (Roswell Park Memorial Institute medium), and PBS (phosphate buffered saline) as well as mixtures thereof at different concentrations. In an embodiment, the shear-thinning can be modified to make the hydrogel injectable.

In an embodiment, the hydrogel of the present invention can be adjusted to have different biophysical and biological properties, including mechanical strength, degradability, and functional ligands can be added to adjust the cell-matrix interaction. Moreover, supplements can be added such as growth factors, and/or small molecules can be added to the cell suspension before mixing with hydrogel to generate a gel-cell mixture formulation.

In an embodiment, the present invention relates to a hydrogel bead system that can make cell spheroids inside of the beads, but can also produce cells that are attached to and/or extended inside of the bead. In an embodiment, one advantage of the present invention over the systems of the prior art is the ease of cell recovery from the beads.

In an embodiment, the hydrogel system of the present invention can have ionic crosslinks, which facilitates recovery. Accordingly, an enzyme-free and/or xeno-free cell recovery solution can be added (for example, a salt solution that allows disruption of ionic crosslinks). In an embodiment, the harvesting can be accomplished at room temperature or at higher temperatures (such as 37° C.) for periods of time without degrading enzymatic systems (because they do not have to be used). By keeping the temperature warm (e.g., 37° C.), molecular exchanges can be accelerated and the harvesting process can be expedited. Very high cell viability and intact 3D structure is maintained. In an embodiment, the isolated cells can be sub-cultured in 2D and 3D cultures after recovery.

In an embodiment, after using an ionic solution to aid in harvesting cells, a solid hydrogel may be converted back to a soft hydrogel. In an embodiment, when the hydrogel is in its soft hydrogel state, it maintains its unique shear thinning properties. In an embodiment, the soft hydrogel can be further transformed from the soft hydrogel state to a liquid hydrogel state wherein with minimal mechanical disruption (such as rocking and/or shaking) and dilution, the cells can be harvested by centrifugation. In an embodiment, harvesting can be attained in about 30 minutes or less, or alternatively 20 minutes or less.

In an embodiment, after the cells have been expanded to a desired level of growth, the cells can be purified/isolated from the hydrogel of the present invention by adding a solution to the cell culture (that also comprises the hydrogel) that dissolves the hydrogel but not the cells. The cells will be suspended in the cell culture while the hydrogel is dissolved. Centrifugation (for example, at 100 g for 3-5 minutes) will pellet the cells at the bottom of a centrifuge tube and the hydrogel will remain with the supernatant. The supernatant containing the hydrogel can be removed and the cells can be washed and/or undergo further processing. For example, the cells may be lysed so as to isolate proteins inside the cell.

Similarly, by selecting an appropriate solvent system, one may be able to isolate exosomes using the same or similar methodologies. For example, the pore size in the hydrogel may be modified so that it allows the passage of smaller molecules but does not allow cells to easily pass through the hydrogel. Accordingly, the hydrogel can be used as a filter to filter the cells, and then using a combination of dissolvability and centrifugation/filtration, one can isolate the small molecules (such as an exosome or a protein of interest).

With the matrix, the attachment of the matrix to the hydrogel is important.

The hydrogel matrix of the present invention more accurately mimics the three-dimensional structure that is present in in vivo systems.

The prior art relied on the surface area. There is limited space to expand in the systems of the prior art. Because, the prior art depends on surface area, if one wanted to try to adapt the two-dimensional systems to three dimensions, the prior art had to coat the cells with chemical adherents, which does not adequately mimic in vivo conditions.

In an embodiment, one key to the present invention is to have the hydrogel matrix structure to support cell attachment.

The system contains 70-95% water, or in an alternate embodiment, from about 90-95% water.

By having the hydrogel structure inside, the surface area problem of the prior art can be solved. The system of the present invention does not need an adherent.

Advantages to the present invention include at least the following. The present system has the ability to be grown in a bioreactor system. The cells can be harvested under gentler parameters thereby reducing cell shear. This allows for gentler conditions to be used, as the cells do not have to be removed/excised from the hydrogel structure. The hydrogel can be dissolved by solvents that are relatively mild that do not disrupt the cell structure and can be at least partially purified using mild purification steps such as centrifugation (at relatively low speeds for short periods of time) and filtration. The use of the hydrogel beads as disclosed herein is easier to scale up. The hydrogel beads as disclosed herein can be used in conjunction with bioreactors, thereby allowing relatively large quantities to be made rather inexpensively. The hydrogel beads as disclosed herein can grow cells at amounts that are 70 to 95% higher than the cells that are grown in the prior art. In an alternate embodiment, the cells can be grown at amounts that are 80 to 95% higher than the cells that are grown in the prior art. Thus, the technology of the present invention allows more cells to be grown at a lower cost. The technology of the present invention can also be used to produce and isolate exosomes and/or cytokines, at high concentrations. Proteins can also be expressed at higher concentrations more efficiently.

In an embodiment, the hydrogel systems of the present invention can be used for a bioreactor scale-up by either using the hydrogel-cell bead method or the static suspension culture method. In an embodiment, and for both of these methods, the cells and hydrogels can be mixed (in a bead shape) and/or the cells can be suspended homogeneously in a hydrogel as a mixture. The hydrogel matrix supports cell attachment in these methods and the beads/gel mixture can be cultured in a bioreactor system for a relatively easy scale up process.

One key advantage of the present invention is that the gel supports cell attachment. A functional integrin ligand such as an RGD peptide can increase the cell-matrix interaction and improve cell attachment. Accordingly, this factor is one of the keys that allows for superior adherent growth relative to the prior art technologies.

Other advantages include the fact that hydrogel technology does not rely on the surface area to grow cells. In cell therapy, the cells need to be as free as possible and cells of the present invention may not be attached to the hydrogel. The cells can be grown in a manner that allows for the cell number to be easily increased. Moreover, the present invention allows for one to culture cells for long periods of time without passing them on to the next phase of cell growth. An additional advantage is that cells can be made as needed. Moreover, harvesting is easier using the hydrogel system of the present invention. When grown in two dimensions, cells need to be discharged by chemical means from the surface in order to wash the cells. The three-dimensional technology of the present invention does not require the cells to be discharged to wash them.

One further advantage is that the hydrogels of the present invention present highly reproducible systems so that one can consistently grow cells in comparable amounts, and can present the system with reproducible systems that accurately mimic in vivo systems.

In an embodiment, the hydrogel can be coated. In a variation, the hydrogel can be manipulated to have different pore sizes. In a variation, the hydrogels can also have functional ligands attached to them.

In an embodiment, when the hydrogel beads of the present invention are used, the release point can be accurately controlled.

In an embodiment, the present invention relates to improved recovery methods, and also to improved harvesting.

The hydrogel systems of the present invention are easy and versatile. The hydrogel solution of the present invention can be mixed with a cell suspension at room temperature, the operation process can be done in 20 minutes, which includes a 10-15 min waiting time for hydrogel stabilization. In an embodiment, the mechanical properties of the hydrogel can be tuned by adjusting the hydrogel concentration with a dilution solution that allows the hydrogel to be modified. Biological functional ligands (such as RGD, collagen, and laminin) can be added to the hydrogels of the present invention so as to more closely mimic the in vivo natural extracellular matrix (ECM) environment. MMPs (metal metalloproteinases) may also be added at some point to the hydrogel bead. The MMPs can be used to degrade the hydrogel systems, which can aid in solubilizing the hydrogel after the hydrogel bead is used for cell growth. Accordingly, the hydrogel systems of the present invention can be tailored to create the 3D culture micro-environment desired by blending different components at different times into the systems to be used for different applications. For example, drug screening, immunofluorescence analysis, and cytotoxicity assays can be performed in the hydrogel of the present invention. Cells cultured in the hydrogel system of the present invention can be easily harvested using solutions that are made for organoid recovery for downstream analysis or subculture.

In an embodiment, the present invention relates to a xeno-free (animal origin-free) hydrogel system that can support three-dimensional (3D) cultures of mesenchymal stem cells (MSCs) or other cell systems. This hydrogel system can be used to make hydrogel cell beads for scale-up. In a variation, microcarriers are not required for MSC scale-up.

In an embodiment, the present invention relates to using a MSC ready-to-use optimized formulation that fully supports the rapid expansion of MSCs along with the hydrogel beads of the present invention. In an embodiment, cells can be directly thawed from liquid nitrogen or passaged from a 2D culture vessel and optionally be mixed (e.g., immediately) with the hydrogel solution for a hydrogel-cell bead generation. The solution hydrogel system in an embodiment is compatible with most MSC culture media and tissue culture vessels. Subsequently, the cells after growth, can be isolated using another cell recovery solution, and cell harvesting can be accomplished after 3D cell culture growth. Thus, the hydrogel bead system of the present invention is both simple and efficient.

In an embodiment, functional ligands can be added to the hydrogel bead systems of the present invention. These functional ligands perform important roles for in vitro cell culture that allow one to accurately mimic their in vivo counterparts. Different functional adhesive ligands can be used, such as RGD, collagen, and/or Laminin. Since these functional ligands are the same or mimic the functionality of in vivo systems, scientists can combine and vary the concentrations of the individual functional ligands to form a heterogeneous customized microenvironment that allows one to optimize the cell growth or optimize the production of the desired small molecule (e.g., exosomes).

In an embodiment, different mixes of hydrogels can be combined together. Generally, the hydrogels of the present invention may be combined with other naturally occurring hydrogels such as collagen or fibrin. Alternatively, the hydrogel beads of the present invention can be combined with other hydrogel compounds such as fibrins, collagens, hyaluronic acid, polypeptides, polyethylene glycol, and/or polyacrylamides.

In an embodiment, naturally occurring compounds can be mixed with the hydrogel system of the present invention. In an embodiment, these compounds may be xeno-free (for example, they will not contain fetal bovine serum). The compounds may be compounds that stabilize the cells and keeps them from shrinking. In an embodiment, the compounds include but are not limited to heparin, fibronectin, and/or human collagen (for example, human collagen IV).

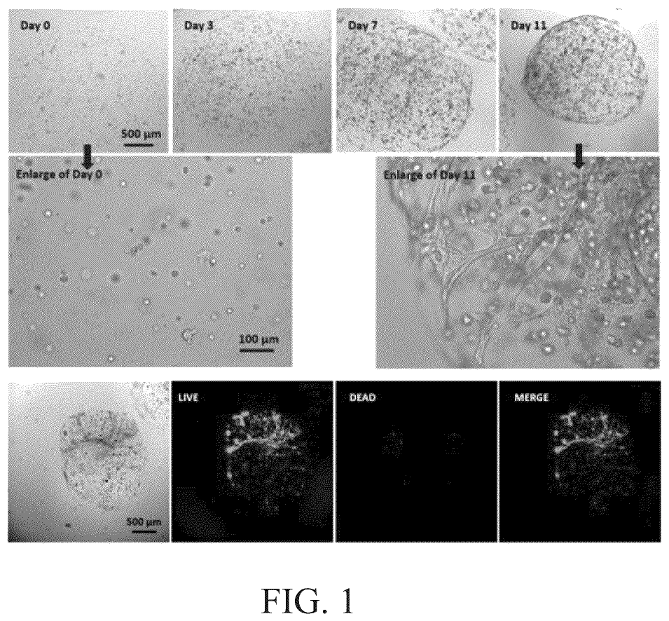

FIG. 1 shows an example of the rapid expansion rate and the health of cultured MSCs using the systems of the present invention over a period of 11 days. In FIG. 1, the hydrogel system of the present invention can produce twice the number of cells and exosomes per ml with much higher viability than traditional microcarriers or 2D culture systems.

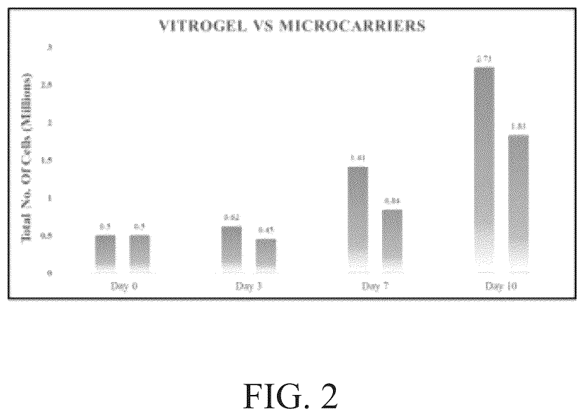

FIG. 2 shows a bar graph of the hydrogels of the present invention (blue bars) vs. the microcarriers of the prior art (orange bars) over a period of 11 days. As can be seen from FIG. 2, the growth at day 0 is even with the microcarriers of the prior art. However, at days 3, 7 and 10, the present invention has an acceleration of growth that far exceeds the microcarriers of the prior art.

Table 1 below shows how hydrogels of the present invention produce superior properties relative to the microcarriers of the prior art including a higher concentration of particles and better protein concentrations. VitroGel is a xeno-free synthetic polysaccharide-based hydrogel system, and it is this polysaccharide-based hydrogel that allows for one to attain the superior properties as disclosed herein.

TABLE 1

| Parameters | Vitrogel Method | Microcarrier Method |
|---|---|---|
| Average diameter (nm) | 98.1 | 93.7 |
| Concentration (Particles/ml) | 2.8E+11 | 2.0E+11 |
| Zeta Potential @ 25° C. (mV) | −31.67 +/− 0.33 | −9.63 +/− 0.10 |
| Protein Concentration | 6.43 | 5.20 |
| (mg · ml) | | |

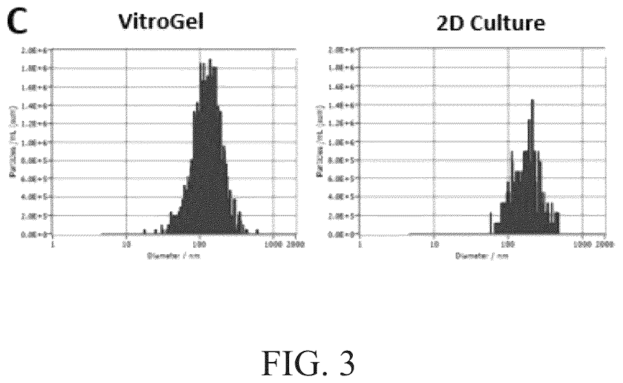

FIG. 3 shows a bar graph showing the relative number of cells in a cell expansion of the present invention versus a 2D culture. As can be seen in FIG. 3, the hydrogels of the present invention provide increased growth relative to the 2D cultures of the prior art.

The following Table 2 illustrates quantitatively how cells grown using the hydrogels of the present invention have superior properties relative to the 2D cultures of the prior art including a higher concentration of particles.

TABLE 2

| Parameters | Vitrogel Method | 2D Culture Method |
|---|---|---|
| Average diameter (nm) | 130.77 | 170.56 |
| Concentration (Particles/ml) | 7.8E+10 | 3.9E+10 |
| Zeta Potential @ 25° C. (mV) | −15.76 +/− 0.09 | −15.33 +/− 0.33 |

In an embodiment, the present invention relates to a method of producing and at least partially purifying exosomes and/or cytokines using a hydrogel bead, said method comprising: encapsulating cells in the hydrogel bead, growing the cells to a level where the cells produce exosomes and/or cytokines, using the hydrogel bead to filter the exosomes and/or cytokines thereby at least partially purifying the exosomes and/or cytokines. In an embodiment, the hydrogels of the present invention can employ additional ingredients such as alginate containing hydrogels.

In an embodiment, the hydrogel bead is around 50-200 μm in size. Having hydrogel beads less than about 300 μm allows them to be used for widespread applications, including being used in the medical, pharmaceutical, and bioengineering fields. Hydrogel beads of this size are advantageous because they allow better transport of nutrients, better dispersion characteristics, better mechanical strength, and allow for easier implantation.

In an embodiment, the concentration of particles (for examples, exosomes) can be determined by nanoparticle tracking analysis, which uses light scattering and Brownian motion to measure both the size and distribution of nanoparticles. A laser beam illuminates the sample of particles in a liquid suspension and the scattering of the light is captured by a camera that allows an estimation of size and concentration of the particles via standard curves and software associated with the nanoparticle tracking analysis. In a variation, the diffusion characteristics/coefficients of the particles can be measured, which also allows for a relatively accurate estimation of the size of the nanoparticles.

In an embodiment, the hydrogels of the present invention are able to encapsulate cells and other hydrogels can be used to make the gel shell. Thus, in one embodiment, if one uses different hydrogels, controlled release becomes superior because different systems can be used to release the cells as required. By varying the relative amounts of the various materials that are used to make the hydrogels (such as in layers), the release can become more tunable than by using only a single type of hydrogel.

In an embodiment, the hydrogel bead further comprises another hydrogel component. In a variation, the alginate containing hydrogel further comprises one or more members selected from the group consisting of: collagen, fibrin, hyaluronic acid, polyacrylamide, polyethylene glycol, and polypeptides. In a variation, the hydrogel further comprises one or more members selected from the group consisting of: collagen, fibrin, hyaluronic acid, polyacrylamide, polyethylene glycol, and polypeptides that form a copolymer, or a terpolymer with the alginate containing hydrogel.

In a variation of the method, the method further comprises a step of dissolving the hydrogel in a solvent system. In a variation, a crosslinker can be added to the hydrogel bead. In a variation, the crosslinker is calcium chloride.

In an embodiment, the method further comprises a step of adding an adhesive ligand to the hydrogel bead. In a variation, the adhesive ligand comprises one or more of RGD, collagen, and/or laminin functional ligands.

In an embodiment, the method further comprises a step of at least partially isolating and purifying the cells. In a variation, the cells are MSCs. In a variation, the cells are grown in a bioreactor.

In an embodiment, the present invention relates to expanding and at least partially purifying cells using a hydrogel bead, said method comprising: encapsulating the cells in the hydrogel bead in a hydrogel matrix, growing the cells, using the hydrogel bead to filter small molecules thereby at least partially purifying the cells, dissolving the hydrogel with a solvent that leaves the cells suspended in the solvent and/or releases them from the hydrogel matrix, centrifuging or filtering the cells to at least partially purify the cells.

In a variation, the cells are MSCs. In a variation, the cells are grown to a concentration of at least 10⁵ cells/ml. In a variation, the cells are grown to a concentration of at least 10⁶ cells/ml. In a variation, the cells are grown to a concentration of at least 10⁷ cells/ml. In a variation, the small molecules comprise exosomes and/or cytokines.

In an embodiment, the present invention allows for a plurality of cell densities to be created. In an embodiment, single cell encapsulation methodologies can be practiced.

In a variation, the centrifuging is performed at 1 g for at least 3 minutes. In a variation of the method, the solvent comprises MPPs and/or sodium citrate. In a variation, the hydrogel bead is an alginate containing hydrogel.

All references cited herein are incorporated by reference in their entireties for all purposes. Other references that are incorporated by reference in their entireties include:
Jin Cheng, Yixin Sun, Yong Ma, Yingfang Ao, Xiaoqing Hu, and Qingyang Meng; Engineering of MSC-Derived Exosomes: A Promising Cell-Free Therapy for Osteoarthritis. Membranes (Basel). 2022 August; 12(8): 739.
WO2022144489 A1
US20220218756 A1
WO2021019562 A3
WO2017087500 A1
US20220220561 A1
U.S. Ser. No. 11/111,475 B2
US20210401895 A1

It should be understood, and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A method of using a 3D hydrogel system to support scale-up and bioproduction of cells; said method comprising utilizing a 3D hydrogel wherein the cells are mixed and/or encapsulated in a hydrogel matrix, and grown in the hydrogel matrix.

2. The method of claim 1, wherein one or more functional ligands and/or small molecules can be added to the 3D hydrogel to generate a modified hydrogel.

3. The method of claim 2, wherein the modified hydrogel increases a cell-matrix interaction to support improved adherent cell attachment.

4. The method of claim 2, wherein the hydrogel system further comprises a different hydrogel and/or supplements.

5. The method of claim 1, wherein the hydrogel system supports exosome(s) and/or protein(s) production.

6. The method of claim 5, wherein the hydrogel system further acts as a filter system for isolation and purification of the exosome(s) and/or the protein(s).

7. A method of producing and at least partially purifying exosomes and/or cytokines using a hydrogel bead, said method comprising:
encapsulating cells in the hydrogel bead,
growing the cells to a level where the cells produce exosomes and/or cytokines,
using the hydrogel bead to filter the exosomes and/or cytokines thereby at least partially purifying the exosomes and/or cytokines.

8. The method of claim 7, wherein the hydrogel bead produces exosomes at higher levels relative to microcarriers or 2D methodologies.

9. The method of claim 8, wherein the hydrogel bead further comprises another hydrogel component.

10. The method of claim 8, wherein the hydrogel further comprises one or more members selected from the group consisting of: collagen, fibrin, hyaluronic acid, polyacrylamide, polyethylene glycol, and polypeptides.

11. The method of claim 1, further comprising a step of dissolving the 3D hydrogel in a solvent system.

12. The method of claim 7, further comprising a step of adding an adhesive ligand to the hydrogel bead.

13. The method of claim 12, wherein the adhesive ligand comprises one or more of RGD, collagen, and/or laminin functional ligands.

14. The method of claim 7, further comprising the step of at least partially isolating and purifying the cells.

15. The method of claim 1, wherein the cells are adhesion cells, suspension cells, tissue segments, and/or a mixture of different types of cells.

16. A method of expanding cells to produce exosomes and/or cytokines using a hydrogel bead, said method comprising:
encapsulating the cells in the hydrogel bead in a hydrogel matrix,
growing the cells,
allowing the cells to produce exosomes and/or cytokines.

17. The method of claim 16, further comprising
using the hydrogel bead to filter small molecules thereby at least partially purifying the exosomes and/or cytokines from the cells.

18. The method of claim 17, further comprising
dissolving the hydrogel bead with a solvent that release the cells from the hydrogel matrix,
centrifuging or filtering the cells to at least partially purify the exosomes and/or cytokines from the cells.

19. The method of claim 18, wherein the cells are one or more of adhesion cells, suspension cells, tissue segments, and/or a mixture of different types of cells.
